(19) United States
(12) Patent Application Publication
(10) Pub. No.: US 2006/0159780 A1
(43) Pub. Date: Jul. 20, 2006

(54) Prevention and amelioration of acetaminophen toxicity with tea melanin

(75) Inventor: Guewha Steven Huang, Hsinchu (TW)

Correspondence Address: BUCKNAM AND ARCHER, 1077 NORTHERN BLVD., ROSLYN, NY, 11576, US

(73) Assignee: National Chiao Tung University

(21) Appl. No.: 11/098,056

(22) Filed: Apr. 4, 2005

(30) Foreign Application Priority Data
Jan. 18, 2005 (TW) 094101377

(51) Int. Cl. A61K 36/82

(52) U.S. Cl. 424/729

(57) ABSTRACT

The present invention relates to a method for preventing and/or reducing the toxicity of acetaminophen which comprises administering to a mammal an amount of tea melanin before or simultaneous with dosage of acetaminophen, a pharmaceutical composition containing tea melanin, and a pharmaceutical composition containing tea melanin and acetaminophen for preventing and/or reducing the toxicity of acetaminophen.

### FIELD OF THE INVENTION

The present invention relates to the field of a method for preventing and/or reducing the toxicity of acetaminophen, and a pharmaceutical composition containing tea melanin and acetaminophen for preventing and/or reducing the toxicity of acetaminophen.

### BACKGROUND OF THE INVENTION

N-Acetyl-p-aminophenol (NAPAP) is a common analgesic and antipyretic drug also known as Acetaminophen, Paracetamol, or Tylenol. NAPAP is safe in therapeutic doses; however, the overdosing can produce hepatic injury causing centrilobular hepatic necrosis, liver failure, and death (see Ray, S. D. et al., J. Pharmacol. Exp. Ther. 1996, 279, 1470-1483). Severe poisoning with NAPAP often requires liver transplantation (see Koivusalo, A. M. et al., Duodecim 2002, 118, 649-650). The production of antibodies also is inhibited by NAPAP (see Yamaura, K. et al., Biol. Pharm. Bull. 2002, 25, 201-205).

Hepatotoxicity of NAPAP is greatly associated with accumulation of the highly reactive metabolite, N-acetyl-p-benzoquinoneimine (NAPQI) generated by cytochrome P450 monooxygenase (see Ray, S. D. et al., J. Pharmacol. Exp. Ther. 1996, 279, 1470-1483 and Esterline, R. L. et al., Biochem Pharmacol. 1989, 38, 2387-2390). NAPQI is normally detoxified by conjugation with reduced gluthatione (GSH). However, when the capacity of GSH is exhausted, NAPQI covalently binds to other cellular structures resulting in hepatic injury (see Albano, E. et al., Mol. Pharmacol. 1985, 28, 306-322). Suppression of P450 can decrease the formation of reactive metabolite and, therefore, may be useful for protection against NAPAP hepatotoxicity (see Jeong, H. G. et al., Biochem. Mol. Biol. Intl. 1998, 45, 163-170 and Jorgensen, L. et al., Pharmacol. Toxicol. 1988, 62, 267-271).

Oxidative stress and, particularly, lipid peroxidation also contribute in the progression of hepatic injury induced by NAPAP (see Wendel, A. et al., Biochem. Pharmacol. 1979, 28, 2051-2055). It was demonstrated that the loss of endogenous antioxidants played a critical role in NAPAP-induced hepatic injury (see Amimoto, T. et al., Free Radic. Biol. Med. 1995, 19, 169-176). Therefore, replenishing antioxidants can serve as a preventive measure against the hepatotoxicity of NAPAP. From this point, the natural antioxidants presented in food might be of particular interest (see Stavric B. Clinical Biochemistry 1994, 27, 319-332). For example, U.S. Pat. No. 5,260,340 has disclosed the use of □-carotene to prevent or reduce NAPAP toxicity, and U.S. Pat. No. 5,670,549 has disclosed the use of □-carotene to treat liver toxicity. In addition, the antioxidants contained in tea have been found to be promising natural protectors (see Katiyar, S. K. et al., Cancer Letters 1993, 73, 167-172. and Wang Z. Y. et al., Carcinogenesis 1989, 10, 411-415). Tea was the oldest folk medicine known in China 5000 years ago due to its detoxifying properties. The major composition and properties of tea antioxidants are well documented, but scarce information is available concerning their polymeric forms (Balentine, D. A. et al., Critical Rev. Food Sci. Nutrition 1997, 37, 693-704).

Recently, the melanin from Thea sinensis Linn has been extracted (see Sava, V. M. et al., Food Chemistry 2001, 73, 177-184.). Similar melanin pigments derived from various sources were intensively studied earlier (see Nicolaus, R. Melanins, Hermann; Paris, France, 1968. and Prota, G. Melanins and melanogenesis, Academic Press; San Diego, 1998.), and the most significant properties concerning melanin chelating and free radical properties were disclosed. Thea sinensis melanin (hereinafter referred to as “TSM”) represents the high molecular part of tea polyphenols (see Hung, Y.-C. et al., J. Ethnopharmacol. 2002, 79, 75-79) with physico-chemical characteristics that are similar to those of typical melanin. TSM has demonstrated a wide range of biochemical and pharmacological activities in animals including antioxidant, free radical scavenging, and immunomodulatory effects (see Sava, V. M. et al., Food Chemistry 2001, 73, 177-184.; Sava, V. M. et al., Food Res. Int. 2001, 34, 337-343.; and Hung, Y-C. et al., Food Chemistry 2002, 78, 233-240). TSM also revealed unexpected protective activity against various toxic substances such, as benzidine, hydrazine and snake venoms (see Sava, V. M. et al., Food Res. Int. 2002, 35, 619-626; Sava, V. M. et al., Food Res. Intl. 2003, 36, 505-511.; Hung, Y-C. et al., Life Sci. 2003, 72, 1061-1071.; and Hung, Y-C. et al., Life Sci. 2004, 74:2037-2047).

On the other hand, present methods of treating acetaminophen overdose include induction of vomiting, stomach lavage, and/or administration of acetylcysteine to replenish hepatic glutathione. These methods, while somewhat effective in preventing injury if performed within 24 hours of ingestion of the excess amount, are not preventative measures that can inhibit toxicity from the initial time of ingestion. These methods also usually require assistance of medical personnel, which might not be immediately available, particularly because acetaminophen is available for use without a prescription.

There remains a need for an effective method to prevent and/or reduce acetaminophen toxicity in a convenient manner, without need for medical supervision or assistance. Therefore, one purpose of the present invention is to examine whether tea melanin could work against overdoses of NAPAP and could work on the reticuloendothelial system of the liver based on the immunostimulating properties (see Sava, V. M. et al., Food Res. Int. 2001, 34, 337-343 and Kate, K. et al., J. Hepatol. 1995, 23, 81-94). The present invention fulfills this need and provides further related advantages.

### SUMMARY OF THE INVENTION

In one aspect, the present invention provides a method for preventing or reducing the toxicity of acetaminophen in a mammal in need of such treatment, said method comprising administering tea melanin to said mammal in an amount effective to prevent or reduce the toxicity of the excessive quantity of acetaminophen.

In another aspect, the present invention provides a pharmaceutical composition for preventing or reducing the toxicity of acetaminophen in a mammal, comprising tea melanin as an active ingredient and a pharmaceutically acceptable carrier or excipient.

In a further aspect, the present invention provides a pharmaceutical composition for preventing or reducing the toxicity of acetaminophen in a mammal, comprising acetaminophen and tea melanin; and a pharmaceutically acceptable carrier or excipient.

### BRIEF DESCRIPTION OF THE DRAWINGS

FIG. 1 shows effect of various doses of TSM given to mice 2 h prior NAPAP administration (400 mg/kg) on accumulation of TBARS in liver tissue. Results are expressed as mean±SEM of six experiments. The C bar depicts the TBARS level in the control group not receiving any treatment. The NC bar represents the negative control, the PC bar represents the positive control, and numbers indicate doses of TSM (in mg/kg). Asterisks depict significant differences between the positive control and the joint effect of NAPAP and TSM (*P<0.05, **P<0.01).

FIG. 2 shows effect of various doses of TSM on SOD activity in liver of ICR mice poisoned with NAPAP. Results are expressed as mean±SEM of six experiments. The C bar depicts SOD activity in the control group not receiving any treatment. The NC bar represents the negative control, the PC bar represents the positive control, and numbers indicate doses of TSM (in mg/kg). Asterisks depict significant differences between the positive control and the joint effect of NAPAP and TSM (*P<0.05, **P<0.01).

FIG. 3 shows effect of various doses of TSM on the content of endogenous CoQ₉ (filled bars) and CoQ₁₀ (open bars) in the livers of mice administered with 400 mg/kg of NAPAP. Results are expressed as mean±SEM of six experiments. The C bars depict coenzyme Q₉ and Q₁₀ levels in the control group not receiving any treatment. The NC bars represent the negative control, the PC bars represent the positive control, and numbers indicate doses of TSM (in mg/kg). Asterisks depict significant differences between the positive control and the joint effect of NAPAP and TSM (*P<0.05, **P<0.01).

FIG. 4 shows effect of TSM on antibody-producing response of splenocytes of ICR mice. Squares depict the effect of TSM itself, and circles represent the joint effect of TSM+NAPAP. The dashed line represents the antigen control. Mice were sensitized with SRBC one day after administration of TSM and/or NAPAP. The mean and SEM (error bars) was obtained from four animals. Asterisks depict significant differences between the antigen control and the effects of NAPAP and TSM (*P<0.05, **P<0.01).

### DETAILED DESCRIPTION OF THE INVENTION

The author of the present invention has discovered the inhibitory effect of tea melanin on cytochrome P450 that was considered to be especially beneficial against hepatotoxicity of NAPAP, and the effect of restoring immunity that was suppressed by NAPAP.

One object of this invention is to prevent or reduce the toxicity of overdosing NAPAP in a mammal, especially the liver toxicity, by using the antioxidant properties, inhibitory effect on cytochrome P450, and immunomodulatory effects of the tea melanin.

A further object of this invention is to prevent and/or reduce the toxicity of NAPAP in a mammal, via administrating tea melanin to block major pathways of acetaminophen-induced liver toxicity. Tea melanin administration is therefore useful, for example, in allowing persons to take higher doses of acetaminophen, and receive the subsequently greater therapeutic benefit without the severe risk of suffering from its toxic effects. Tea melanin is also useful for preventing damage to individuals who have a special sensitivity to the toxic effects of acetaminophen, such as alcoholics.

In one embodiment of the present invention, TSM (10-40 mg/kg) effectively decreased the mortality of NAPAP (400 mg/kg) toxicity. TSM has protective effects against the hepatic injury induced by NAPAP and also significantly reduces NAPAP-induced hepatic GSH depletion. The protective effects were evidenced by determination of ALT activity and GSH and GSSG assays. In a further embodiment, TSM caused a dose-dependent inhibition of cytochrome P450 isozymes 2E1, the suppression of P450 activity could result in the decreased formation of the reactive metabolites NAPQI causing liver injury, decrease the conjugation of NAPQI with GSH, and therefore prevent depletion of GSH. According to the two embodiments of the present invention, tea melanin caused an inhibitory effect on the activity of hepatic isozymes of cytochrome P450 2E1 and significantly reduced NAPAP-induced hepatic GSH depletion.

In one embodiment of the present invention, TSM (10-40 mg/kg) significantly decreased NAPAP-induced lipid peroxidation in a dose-dependent manner. In a further embodiment, TSM also caused activation of SOD. Still in one embodiment of the present invention, TSM recovered content of endogenous coenzymes Q₉ and Q₁₀. According to these embodiments of the present invention, the antioxidant activity of tea melanin reduced the cellular oxidative stress caused by NAPAP and therefore protected animals against NAPAP toxicity.

In a further embodiment, the immune suppressive effect of NAPAP on the in vivo antibody-forming cells (AFC) responses was demonstrated using ICR mice sensitized with SRBC. The joint effect of TSM and NAPAP indicated the capability of TSM to restore the immunity of the animals to the level of intact mice. According to this embodiment of the present invention, the immunostimulation activity of tea melanin protected animals against immunosuppression induced by NAPAP.

The present invention demonstrates that the protective effect of tea melanin against NAPAP hepatotoxicity is based on a combination of different factors including cytochrome P450 inhibitory activity, antioxidant properties, and immuno-stimulation. Such a combination opens the possibility for a comprehensive protection of the liver against heavy intoxication and may serve as a first approach in developing natural hepatoprotectors.

According to the present invention, a method for preventing or reducing the toxicity of acetaminophen in a mammal in need of such treatment is provided, comprising administering tea melanin to said mammal in an amount effective to prevent or reduce the toxicity of the excessive quantity of acetaminophen, wherein the tea melanin can be administered either before, simultaneous with, and/or after ingestion of the acetaminophen.

In one aspect of the present invention relates to a pharmaceutical composition for preventing or reducing the toxicity of acetaminophen in a mammal, comprising an effective amount of tea melanin as an active ingredient.

Still in one aspect of the present invention relates to a pharmaceutical composition containing acetaminophen for preventing or reducing the toxicity of acetaminophen in a mammal, comprising an effective amount of tea melanin as an active ingredient.

According to the method of the present invention, the effective amount of tea melanin is preferably in the range from about 10 to about 200 mg of tea melanin per day for a 70 kg human, or from about 0.1 to about 3 mg/kg of body weight. According to the pharmaceutical composition of the present invention, the effective amount of tea melanin is preferably in the range from about 0.1 to about 3 mg/kg of body weight.

According to the present invention, the term “mammal” as used herein includes a human being. Preferably, the mammal is a human.

For therapeutic administration, the pharmaceutical composition of the present invention are used in the form of a conventional pharmaceutical preparation in admixture with a conventional pharmaceutically acceptable carrier, including but not limited to tablet, capsule, powder, solution, or suspension.

The pharmaceutical composition of the present invention can be administered by oral, parenteral, or intraperitoneal administration, including but not limited to intravenous and intramuscular administration. Additionally, according to the method of the present invention, tea melanin and acetaminophen can be formulated separately, or in a combined formulation, in any form suitable for the desired route of administration.

The following examples are provided to illustrate the invention and do not limit the scope thereof. One skilled in the art will appreciate that although specific reagents and conditions are outlined in the following examples, modifications can be made which are meant to be encompassed by the spirit and scope of the invention.

Materials and Methods

Materials.

The Thea sinensis melanin (TSM) is used in all assays hereinto. Thea sinensis leaves were harvested in Miaoli, Taiwan and were identified in the Institute of Chinese Pharmaceutical Sciences, China Medical University, and a voucher specimen (GSH-001) was deposited at the Herbarium of this Institute. NAPAP, EDTA, Tris-HCl, Triton X-100, Sephadex G-75, molecular size markers, and a kit for serum alanine aminotransferase activity were purchased from Sigma Chemical Co. (St. Louis, Mo.). All other chemicals were of analytical grade or higher from Merck (Darmstadt, Germany).

Isolation and Physico-Chemical Characterization of TSM

Isolation of TSM was performed according to the previously reported procedure (Sava, V. M. et al., Food Chemistry 2001, 73, 177-184) employed with minor adjustments. Namely, the extraction time was diminished to 12 h avoiding excessive oxidation of TSM. After extraction, the mixture was filtered and centrifuged at 15,000 g for 30 min to obtain TSM extract. This extract was acidified by the addition of 2N HCl to pH 2.5 and centrifuged at 15,000 g for 15 min to pellet form. Acid hydrolysis was employed for purification of TSM. The purified product thus obtained was dissolved in 0.2% NH₄OH, and the solution was subjected to repeated precipitations. Precipitation procedure was repeated for additional three times to sequester TSM from low molecular impurities and to improve its homogeneity. The solutions thus obtained were filtered through a Nalgene 0.45 μm syringe filter. Finally, TSM was purified on a Sephadex G-75 (the column's dimensions were 1.6×40 cm) in a 50 mM phosphate buffer (pH 7.5) at a flow rate of 0.5 ml·min⁻¹. Fractions were monitored at 280 nm. To evaluate the molecular mass (MM) of TSM, a Sephadex G-75 column was calibrated with bovine serum albumin (MM 66,000), carbonic anhydrase (MM 29,000), cytochrome C (MM 12,400), and aprotinin (MM 6,500) as size markers.

Physical and chemical characterizations of TSM were performed according to conventional procedures (Nicolaus, R. Melanins, Hermann; Paris, France, 1968. and Prota, G. Melanins and melanogenesis, Academic Press; San Diego, 1998). Ultraviolet-visible (UV) absorption spectra were obtained with a JASCO V-530 UV-Visible Spectrophotometer (Jasco Ltd., Great Dunmow, UK). Infrared (IR) spectra were recorded for KBr samples on a Perkin-Elmer spectrometer 1600 FT (Perkin-Elmer Instruments, Norwalk, Conn.). Solubility in water, aqueous acid, and in common organic solvents; oxidative bleaching by means of KMnO₄, K₂Cr₂O₇, NaOCl, and H₂O₂; and a positive reaction for polyphenols were used as typical tests for melanin.

Animals and Treatment

Adult male ICR mice (30±5 g) were employed for all experiments. Animals were housed under controlled condition (25±2° C.) and 12 h light/dark cycle, and allowed free access to food and water, but fasted overnight before treatment. Animals were divided into several groups including control group (not receiving any treatment), negative control (receiving TSM alone), positive control (receiving only NAPAP), and experimental groups receiving NAPAP and TSM together. Each experimental group consisted of 6 mice. NAPAP was dissolved in normal saline (pH 7.4) and administered inraperitonially (i.p.) with a dose of 400 mg/kg. TSM was dissolved in distillate water at pH 7.2 and administered i.p. with doses of 10, 20, 30, or 40 mg/kg 2 h before intoxication. All animals were sacrificed by ether anesthesia 24 h after the NAPAP exposure. Blood samples were withdrawn by cardiac puncture for determination of alanine aminotransferase (ALT) activity using commercially available kit (Sigma 505-P). Liver was isolated and perfused with normal saline to wash out the blood. It was then used for determination of GSH, oxidized glutathione (GSSG), superoxide dismutase (SOD), thiobarbituric acid reactive substances (TBARS), and reduced forms of coenzymes Q₉ (CoQ₉) and Q₁₀ (CoQ₁₀).

### EXAMPLE 1

Effect of TSM on the Toxicity of NAPAP: GSH and GSSG Assays and ALT Activity

Frozen liver tissue was homogenized in 5% trichloroacetic acid (TCA) supplemented with 5 mM EDTA under a stream of nitrogen gas, then centrifuged at 20,000 g for 10 min at 4° C. TCA was removed from supernatant by extracting three times with diethyl ether. The total hepatic glutathione level representing the sum of GSH and GSSG was determined using dithiobis-2-nitrobenzoic acid (Griffith, O. W. Anal. Biochem. 1980, 106, 207-212). The GSSG level was determined after elimination of GSH by reaction with 2-vinylpyridine, and the actual GSH level was calculated by subtracting the GSSG level from the total glutathione.

Effect of TSM on the toxicity of NAPAP is summarized in the following Table 1. Administration of 40 mg/kg TSM alone did not induce any toxicity. The behavior of the animals in experimental group resembled the control group by movement activity. In contrary, all animals treated with NAPAP alone (400 mg/kg) were sick as compared to control and were unable to move around the cages. Such treatment caused hepatocellular damage in mice, as indicated by a drastic elevation of ALT level. The animals pretreated with TSM 2 h prior intoxication showed protection against NAPAP. TSM caused a dose-dependent effect against NAPAP challenge, with plasma ALT level being reduced to 74, 14 and 3% of positive control when 10, 20 and 30 mg/kg doses were given to animals. The highest dose of TSM (40 mg/kg) completely blocked hepatotoxicity of NAPAP. Animal lethality observed for 400 mg/kg NAPAP administered alone was 66%. Administration 40 mg/kg of TSM by itself did not give animal death. TSM (10-40 mg/kg) administered prior NAPAP also prevented lethality of animals.

Hepatic GSH was determined 24 h after the NAPAP administration. TSM, by itself, did not affect hepatic GSH level (Table 1). Administrations of NAPAP alone significantly depleted GSH level (2.6-fold as compared to control). Pretreatment with TSM decreased GSH depletion demonstrating a dose-dependent protective effect. Concentration of GSSG was restored to about the same level suggesting that GSH loss might not have resulted from reaction with glutathione peroxidase, but was due to conjugation glutathione with NAPQI.

TABLE 1
Effect of TSM on toxicity of NAPAP

| | Mortality | | GSH | GSSG |
|---|---|---|---|---|
| | (dead/ | | (nmol/mg | (nmol/mg |
| Animal groups^{a} | total) | ALT (U/L) | prot.) | prot.) |
| Control | 0/6 | 42 ± 4^{b} | 39 ± 4 | 3.5 ± 0.4 |
| TSM (40 mg/kg) | 0/6 | 40 ± 5 | 40 ± 3 | 3.6 ± 0.3 |
| NAPAP | 2/6 | 2043 ± | 13 ± 1** | 3.2 ± 0.4 |
| (400 mg/kg) | | 231**,^{c} | | |
| TSM (10 mg/kg) + | 0/6 | 1528 ± 142** | 15 ± 2** | 3.5 ± 0.3 |
| NAPAP | | | | |
| (400 mg/kg) | | | | |
| TSM (20 mg/kg) + | 0/6 | 298 ± 30** | 22 ± 2* | 2.9 ± 0.2 |
| NAPAP | | | | |
| (400 mg/kg) | | | | |
| TSM (30 mg/kg) + | 0/6 | 63 ± 5* | 28 ± 3* | 3.3 ± 0.2 |
| NAPAP | | | | |
| (400 mg/kg) | | | | |
| TSM (40 mg/kg) + | 0/6 | 43 ± 5 | 28 ± 2* | 3.1 ± 0.3 |
| NAPAP | | | | |
| (400 mg/kg) | | | | |

^{a}Control mice were given saline. Experimental animals received TSM (10, 20, 30 or 40 mg/kg, i.p.) and NAPAP (400 mg/kg. i.p.) that was injected 2 h after administration of TSM.
^{b}Toxic effect was evaluated at 24 h of NAPAP exposure and data represent means ± SEM.
^{c}Significantly different from the control (*P < 0.05; **P < 0.01).

### EXAMPLE 2

Effect of TSM on Activity of Hepatic Isozymes of Cytochrome P450 Monooxygenase: Evaluation of the Activities of P450 Isozymes

Experiments were performed on 5 groups of intact ICR mice consisting of 6 animals each. Different doses of TSM (0, 10, 20, 30, and 40 mg/kg,) were given to mice i.p. Animals were sacrificed 24 h after injection, livers were removed and perfused with chilled KCl (154 mM), and homogenized in buffer containing 50 mM Tris-HCl and 154 mM KCl (pH 7.4). The homogenates were centrifuged at 9,000 g for 20 min at 4° C., and the microsomal fraction was separated from the supernatant by centrifugation at 105,000 g for 90 min at 4° C. The microsomal pellet was washed with homogenizing buffer, centrifuged again at 105,000 g for 90 min at 4° C. and then suspended in 250 mM sucrose. The activities of ethoxyresorufin O-dealkylase (P450 2A1) and pentoxyresorufin O-alkylase (P450 2B1) were measured according to the method described by Burke and Mayer (Burke, M. D. et al., Biochem. Pharmacol. 1994, 48, 923-36). N-nitrosodimethylamine demethylase activity was determined as an index of P450 2E1 activity by the method of Peng et al. (Peng, R. et al., Carcinogenesis 1982, 3, 1457-1461).

Effect of TSM on activity of hepatic isozymes of cytochrome P450 monooxygenase is summarized in the following Table 2. Pre-administration of TSM caused an inhibitory effect on the activity of hepatic isozymes of cytochrome P450. To determine the relative inhibitory effect on various P450 isozymes, different substrates were used for incubation with liver microsomes. TSM caused a dose-dependent inhibition of P450 2E1 specific N-nitrosodimethylamine demethylase activity with an ED₅₀ value of 15.8 mg/kg body weight. Activities of P450 2A1 and P450 2B1 were changed insignificantly.

TABLE 2
Effect of TSM on activity of hepatic isozymes of cytochrome P450
monooxygenase

| | |
|---|---|
| Experimental | Activity of P450 isozymes^{a} |

| conditions | 2A1 | 2B1 | 2E1 |
|---|---|---|---|
| Control | 7.35 ± 1.23 | 1.4 ± 0.13 | 2.85 ± 0.33 |
| (vehicle) | | | |
| TSM | 7.5 ± 1.41 | 1.6 ± 0.12 | 2.52 ± 0.25 |
| 10 mg/kg | | | |
| TSM | 7.28 ± 1.11 | 1.4 ± 0.15 | 1.51 ± 0.19^{b} |
| 20 mg/kg | | | |
| TSM | 7.44 ± 1.25 | 1.3 ± 0.13 | 1.1 ± 0.09^{b} |
| 30 mg/kg | | | |
| TSM | 7.64 ± 1.52 | 1.5 ± 0.16 | 0.93 ± 0.1^{b} |
| 40 mg/kg | | | |

^{a}Activity is expressed as mean ± SEM of 6 mice. Enzyme activities are expressed as follows: 2A1 and 2B1 - from amount of resorufin (pmol/min · mg protein); 2E1 - from amount of formaldehyde (nmol/min · mg protein)
^{b}Value is significantly different from that of control (P < 0.01).

### EXAMPLE 3

Effect of TSM on NAPAP-Induced Lipid Peroxidation: Determination of TBARS

Formation of lipid peroxide derivatives was evaluated by measuring TBARS according to Cascio et al. (Cascio, C. et al., J. Neurochem. 2000, 74, 2380-2391). Briefly, liver was homogenized in ice-cold 1.15% KCl (w/v); then 0.4 mL of the homogenates was mixed with 1 mL of 0.375% thiobarbituric acid, 15% TCA (w/v), 0.25 N HCl, and 6.8 mM butylated-hydroxytoluene, placed in a boiling water bath for 10 min, removed, and allowed to cool on ice. Absorbance (532 nm) was measured in the supernatants after centrifugation at 3000 r.p.m. for 10 min. The amount of TBARS produced was expressed as nmol TBARS per milligram of protein using malondialdehyde bis(dimethyl acetal) for calibration.

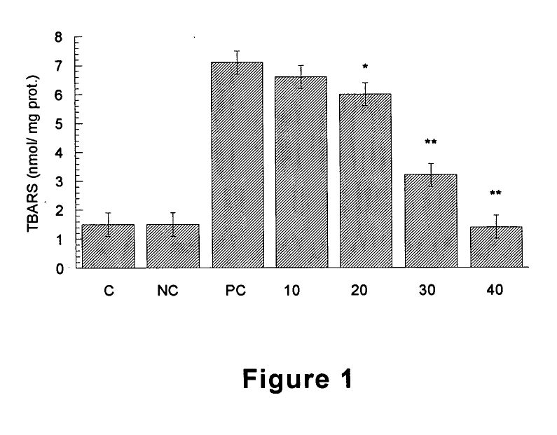

FIG. 1 illustrates the effect of various doses of TSM given to mice 2 h prior NAPAP administration (400 mg/kg) on accumulation of TBARS in liver tissue. Pretreatment by TSM significantly decreased NAPAP-induced lipid peroxidation in a dose-dependent manner. Increased dose of TSM suppressed peroxidation with the highest dose causing full blockage of TBARS. Administration of TSM alone (negative control) did not produce any significant effect as compared to control (no treatments).

### EXAMPLE 4

Superoxide Dismutase Assay

Determination of superoxide dismutase activity in mouse liver was based on inhibition of nitrite formation in reaction of oxidation of hydroxylammonium with superoxide anion radical (Elstner, E. F. and Heupel, A. Anal. Biochem. 1976, 70, 616-620). Nitrite was generated in a mixture containing 25 μL xanthine (15 mM), 25 μL hydroxylammonium chloride (10 mM), 250 μL phosphate buffer (65 mM, pH 7.8), 90 μL distilled water, and 100 μL xanthine oxidase (0.1 U/μL). The inhibitory effect of inherent SOD was assayed at 25° C. during 20 min of incubation with 10 μL of liver tissue extracts. Determination of the resulting nitrite was performed on the reaction (20 min at room temperature) with 0.5 mL sulfanilic acid (3.3 mg/mL) and 0.5 mL α-naphthylamine (1 mg/mL). Optical absorbance at 530 nm was measured with Ultrospec III spectrophotometer (Pharmacia, LKB). The results were expressed as units of SOD activity calculated per milligram of protein.

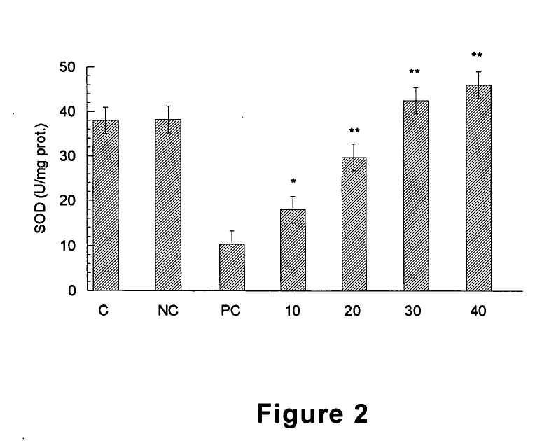

FIG. 2 illustrates the effect of various doses of TSM on SOD activity in liver of ICR mice poisoned with NAPAP. Introduction of NAPAP caused an almost 4-fold decrease of SOD activity as compared to control. Significant restoration of SOD activity was observed when TSM (10-40 mg/kg) was administered 2 h prior to injection of NAPAP. Resumption of SOD activity reached a plateau at higher doses of TSM indicating the ability of TSM to maintain SOD activity at the level of negative control. Administration of TSM alone did not affect SOD activity. This suggests an indirect influence of TSM on SOD activation.

### EXAMPLE 5

Coenzymes Q Assay

Determination of reduced forms of CoQ₉ and CoQ₁₀ was carried out by the method of Ikenoya et al. (Ikenoya, S. et al., Chem Pharm Bull. 1981, 29, 158-164). Liver tissue was homogenized in ice-cold water under a stream of nitrogen gas. Coenzymes Q were extracted with mixture ethanol: n-hexane (2:5 v/v) and n-hexane layer were collected. The solvent was evaporated using a rotary evaporator and re-dissolved in the ethanol. The extract was analyzed using HPLC with Jasco 840 EC detector and Chemosorb ODS-H column (4.6×250 mm). The mobile phase consisted of ethanol:methanol:70% HClO₄ (700:300:1 v/v) supplemented with 0.7% NaClO₄H₂O.

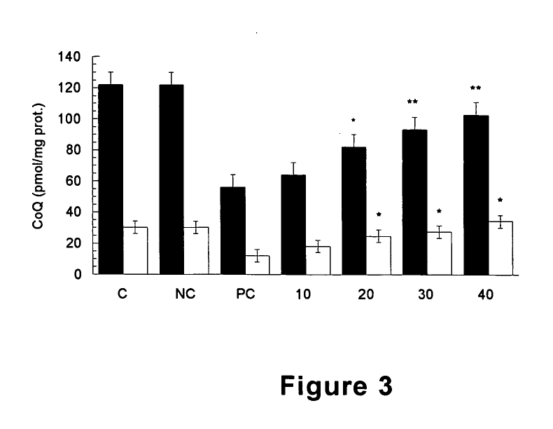

FIG. 3 illustrates effect of various doses of TSM on content of endogenous CoQ₉ and CoQ₁₀ in liver of mice administered with 400 mg/kg of NAPAP. NAPAP itself caused a significant decrease in the quantity of reduced forms of antioxidant enzymes CoQ₉ and CoQ₁₀ in animal liver. Namely, as compared to negative control, coenzyme Q₁₀ content was diminished 55% and the content of CoQ₉ was diminished 60%. Administration of TSM 2 h before NAPAP allowed an increasing quantity of both antioxidant coenzymes in a dose-dependent manner. The highest dose of TSM (40 mg/kg) reduced content coenzyme Q₉ to 83% but increased Q₁₀ to 113% of the control level. However, administering the same dose of TSM alone did not produce any significant difference as compared to control suggesting an indirect effect on coenzymes Q.

### EXAMPLE 6

Evaluation of Antibody-Producing Response

Experiments were performed on 10 groups of intact ICR mice consisting of 4 animals each. The first 5 groups were injected i.p. with the following single doses of TSM: 0, 10, 20, 30, and 40 mg/kg. The second 5 groups were injected similarly to the first groups with TSM, but after 2 h animals were again injected (i.p.) with 400 mg/kg NAPAP. One day after administration of TSM or TSM+NAPAP, mice were injected into the tail vein with 1×10⁸ sheep red blood cells (SRBC) prepared in 0.2 mL of saline. An additional 4 animals received only SRBC (antigen control). Four days after their sensitization with SRBC, animals were sacrificed, and their spleens were removed. Single splenocytes were prepared from each spleen in 5 mL of RPMI-1640 media. Enumeration of the antibody-forming cells (AFC) was performed using a plaque assay (Jerne, N. K. and Nordin, A. A. Science 1963, 140, 405). AFC values were calculated for 10⁶ splenocytes.

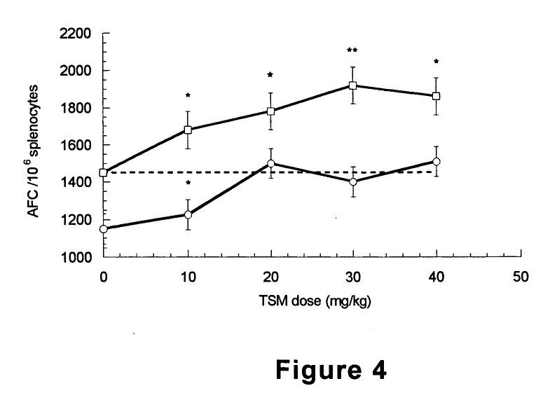

FIG. 4 illustrates effect of TSM on antibody-producing response of splenocytes of ICR mice administered with NAPAP. The in vivo antibody-producing responses were employed to evaluate the effect of TSM alone and the joint effect of TSM+NAPAP on the humoral immunity of ICR mice. Introduction of TSM demonstrated a dose-dependent immuno-stimulating effect similar to that obtained earlier on BALB/C mice (Sava, V. M. et al., Food Res. Int. 2001, 34, 337-343). At doses of 30-40 mg/kg of TSM, the antibody-secreting cells produced significantly (P<0.05) more antibodies (26-28%) than did the antigen control. Administration of NAPAP caused a suppression of AFC of 26% against the antigen control level. However, pre-administration of TSM before NAPAP intoxication increased AFC in a dose-dependent manner, and restored TSM immunity to the level of the antigen control at doses starting from 20 mg/kg.

What is claimed is:

1. A method for preventing or reducing the toxicity of acetaminophen in a mammal in need of such treatment, said method comprising administering tea melanin to said mammal in an amount of effective to prevent or reduce the toxicity of the excessive quantity of acetaminophen.

2. The method according to claim 1, wherein the mammal is a human.

3. The method according to claim 1, wherein the amount of tea melanin administered in the step of administering ranges from about 0.1 mg/kg to about 3 mg/kg of body weight.

4. The method according to claim 1, wherein the step of administering tea melanin occurs before the ingestion of the acetaminophen.

5. The method according to claim 1, wherein the step of administering tea melanin occurs simultaneously with the ingestion of the acetaminophen.

6. The method according to claim 5, wherein the step of administering tea melanin includes a step of administering a composition containing both acetaminophen and tea melanin.

7. The method according to claim 1, wherein the step of administering tea melanin is performed orally.

8. The method according to claim 1, wherein the step of administering tea melanin is performed intravenously.

9. The method according to claim 1, wherein the step of administering tea melanin is performed intramuscularly.

10. A pharmaceutical composition for preventing or reducing the toxicity of acetaminophen in a mammal, comprising acetaminophen and tea melanin; and a pharmaceutically acceptable carrier or excipient.

11. The pharmaceutical composition according to claim 10, wherein the amount of tea melanin is sufficient to prevent or reduce the toxic effects of acetaminophen.

12. The pharmaceutical composition according to claim 10, wherein the mammal is a human.

13. A pharmaceutical composition for preventing or reducing the toxicity of acetaminophen in a mammal, comprising tea melanin as an active ingredient and a pharmaceutically acceptable carrier or excipient.

14. The pharmaceutical composition according to claim 13, wherein the mammal is a human.
